(19) United States
(12) Patent Application Publication
(10) Pub. No.: US 2026/0115052 A1
(43) Pub. Date: Apr. 30, 2026

(54) APPARATUS, SYSTEM, AND METHOD FOR AT LEAST ONE OF ICING, HEATING, AND COMPRESSING A USER'S BODY PART

(71) Applicant: Stephen Eugene Juaire, West Elbow Drive, FL (US)

(72) Inventor: Stephen Eugene Juaire, West Elbow Drive, FL (US)

(21) Appl. No.: 19/005,684

(22) Filed: Dec. 30, 2024

Related U.S. Application Data
(63) Continuation-in-part of application No. 18/130,766, filed on Apr. 4, 2023, now Pat. No. 12,193,915.
Application No. 18/130,766 is a continuation-in-part of application No. 17/941,511, filed on Sep. 9, 2022, now Pat. No. 11,638,468.
(60) Provisional application No. 63/242,398, filed on Sep. 9, 2021.

(51) Int. Cl. A61F 13/04; A61F 7/08; A61F 7/10

(52) U.S. Cl. CPC A61F 13/046; A61F 7/08; A61F 7/10; A61F 2007/108

(57) ABSTRACT

A wearable device for icing, heating, and compressing a user's body part is disclosed. The device comprises an inner part having an upper end and a lower end, and an outer part having an upper end and a lower end. A first attaching means secures the inner part lower end to the outer part lower end, defining a compartment between the inner and outer parts for receiving therapeutic elements. A second attaching means includes a first part attached to an end portion of the inner and outer parts and a second part attached away from the end portion. An extension flap extends beyond the second attaching means, ensuring 360-degree coverage of the compartment. The device forms a tubular body with a central opening, allowing uniform compression and thermal application. A third attaching means secures the extension flap to the device, maintaining consistent coverage and stability during use.

### CROSS-REFERENCE TO RELATED APPLICATIONS

This is a continuation in part application that claims priority to U.S. Non-Provisional application Ser. No. 18/130,766, filed Apr. 4, 2023, which is a continuation in part application of U.S. Non-Provisional application Ser. No. 17/941,511, filed Sep. 9, 2022, which claims priority to U.S. Provisional Application Ser. No. 63/242,398, filed Sep. 9, 2021, and the subject matter both of which is incorporated herein by reference.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present disclosure relates to the field of therapeutic modalities, and more specifically to wearable temperature and compression therapy devices for relieving pain and promoting healing of everyday aches and injuries.

### BACKGROUND

Cold therapy, heat therapy, and compression are widely used therapeutic techniques for managing musculoskeletal injuries and promoting recovery. These therapies aim to alleviate pain, reduce inflammation, and facilitate healing of injured soft tissue. Despite their effectiveness, traditional cold and heat therapy methods often suffer from several limitations that hinder their overall utility and user experience.

One significant limitation of existing therapeutic devices is their inability to provide consistent and comprehensive coverage to an affected body part. Traditional methods, such as ice packs, gel packs, and hot water bottles, are typically rigid or irregularly shaped, making it difficult to maintain contact over curved or jointed areas such as the knee, elbow, or shoulder. Consequently, these modalities often fail to deliver uniform cooling, heating, or compression, which reduces the efficacy of the therapy.

Another challenge with conventional devices is their lack of secure and stable attachment mechanisms. Ice packs or heat pads frequently shift or slip out of position during use, especially if the user engages in movement or physical activity. This problem is exacerbated by the need for secondary securing means, such as straps, bands, or tape, which can be cumbersome to apply and may not provide sufficient stability or comfort. Moreover, these secondary means can create pressure points or gaps, leading to inconsistent compression or therapeutic coverage.

Moisture management is another common issue with existing cold therapy devices. Melting ice or condensation from cold packs can leak onto clothing, causing discomfort and making the devices impractical for prolonged or mobile use. Additionally, direct contact between ice packs and the skin can result in frostbite or thermal burns, necessitating the use of protective layers, which may further reduce the efficacy of the therapy.

Injuries often involve multiple muscle groups or soft tissue areas within close proximity, such as hamstrings, quadriceps, or shoulder muscles. Traditional devices are typically designed to target isolated areas, making it difficult to treat multiple regions simultaneously. This limitation forces users to either alternate between different devices or compromise the effectiveness of their therapy.

Compression therapy, often used in conjunction with cold or heat therapy, presents its own challenges. Existing compression wraps or braces may not offer adequate elasticity or uniform pressure distribution, leading to suboptimal therapeutic outcomes. The need for precise compression that remains consistent during use is critical for preventing edema, stabilizing muscles, and promoting faster recovery.

Therefore, there is a need for an improved therapeutic device that can provide effective and uniform icing, heating, and compression while addressing the shortcomings of traditional methods. Specifically, there is a need for a wearable device that ensures comprehensive coverage, secure attachment, and consistent compression, all while being easy to use, comfortable, and capable of preventing leaks or moisture-related discomfort.

The present invention addresses these needs by providing a wearable therapeutic device with a compartment for ice or heat elements, multiple attaching means, and an extension flap to ensure 360-degree coverage. This device offers a solution to the problems of inconsistent coverage, unstable positioning, and inadequate compression found in existing cold and heat therapy products.

### SUMMARY

An apparatus, system, and method for a wearable device for at least one of icing, heating and compressing a user's body part is disclosed. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a system for at least one of icing, heating and compressing a user's body part is disclosed. The system comprises a wearable device for at least one of icing, heating, and compressing a user's body part is disclosed. The wearable device comprises an inner part having an upper end and a lower end, and an outer part also having an upper end and a lower end. The inner part lower end is attached to the outer part lower end via a first attaching means. A compartment is defined by a space between the outer part and the inner part, positioned above the first attaching means, and designed to hold therapeutic elements such as ice or heat packs. A second attaching means is provided, where a first part of the second attaching means is attached to an end portion of at least the inner and outer parts, and a second part of the second attaching means is attached to at least one of the inner or outer parts away from the end portion. An extension flap extends beyond the second attaching means on at least one of the inner or outer parts, enhancing the seal and coverage of the compartment. The device forms a tubular body with a central opening defined by the removable attachment of the first and second parts of the second attaching means. The compartment surrounds at least a portion of the central opening, and the extension flap ensures complete 360-degree coverage as it extends beyond the second attaching means. This configuration provides effective and uniform compression, as well as thermal therapy, around the entire circumference of the body part, ensuring stability and consistent treatment during use.

Additional aspects of the disclosed embodiment will be set forth in part in the description which follows, and in part will be obvious from the description, or may be learned by practice of the disclosed embodiments. The aspects of the disclosed embodiments will be realized and attained by means of the elements and combinations particularly pointed out in the appended claims. It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute part of this specification, illustrate embodiments of the disclosure and together with the description, explain the principles of the disclosed embodiments. The embodiments illustrated herein are presently preferred, it being understood, however, that the disclosure is not limited to the precise arrangements and instrumentalities shown, wherein:

FIG. 1A is a side perspective side view of an apparatus for at least one of icing, heating, and compressing a user's body part, according to an example embodiment;

FIG. 1B is a side perspective side view of an apparatus for at least one of icing, heating, and compressing a user's body part, according to an example embodiment;

FIG. 2 is a front perspective side view of an apparatus for at least one of icing, heating, and compressing a user's body part, according to an example embodiment;

FIG. 3 is a side cross-sectional view of a system for at least one of icing, heating, and compressing a user's body part, wherein a cold or hot element is located in a first area within the compartment, according to an example embodiment;

FIG. 4 is a side cross-sectional view of a system for at least one of icing, heating, and compressing a user's body part, wherein a cold or hot element is located in a second area within the compartment, according to an example embodiment;

FIG. 5 is a side cross-sectional view of a system for at least one of icing, heating, and compressing a user's body part, wherein a plurality of cold or hot elements are located in different areas within the compartment, according to an example embodiment;

FIG. 6 is a side perspective view of an apparatus for at least one of icing, heating, and compressing a user's body part, wherein a user is positioning a cold or hot element within the compartment, according to an example embodiment;

FIG. 7 is a side cross-sectional view of a system for at least one of icing, heating and compressing a user's body part, according to an example embodiment;

FIG. 8 is a side cross-sectional view of a system of an apparatus for at least one of icing, heating, and compressing a user's body part, according to an example embodiment; and

FIG. 9 is a flowchart illustrating the steps for a method for at least one of icing, heating, and compressing a user's body part, according to an example embodiment;

FIG. 10 is a top view of a diagram of a band, according to an example embodiment;

FIG. 11 is a partial cross-sectional view of a pair of bands attached to the outer part; according to an example embodiment; and

FIG. 12 is a partial cross-sectional view of more clearly illustrating the widths of the outer part and inner part, according to an example embodiment;

FIG. 13A is a top perspective view of an apparatus for at least one of icing, heating, and compressing a user's body part, according to an example embodiment;

FIG. 13B is a top perspective view of an apparatus for at least one of icing, heating, and compressing a user's body part, according to an example embodiment;

FIG. 13C is a top perspective view of an apparatus for at least one of icing, heating, and compressing a user's body part, according to an example embodiment;

FIG. 13D is a side perspective view of an apparatus for at least one of icing, heating, and compressing a user's body part, according to an example embodiment; and

FIG. 13E is a side perspective view of an apparatus for at least one of icing, heating, and compressing a user's body part, according to an example embodiment.

### DETAILED DESCRIPTION

The following detailed description refers to the accompanying drawings. Whenever possible, the same reference numbers are used in the drawings and the following description to refer to the same or similar elements. The figures are drawn to scale. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing uniform pressure and temperature to an unrestricted combination of soft tissue groupings in the human body. In other words, a therapeutic modality is not restricted to a particular location or region on a user's body. The wearable device includes a compartment positioned outward from the user's body for receiving ice defined by a space between an outer part and an inner part of the wearable device. The space may vary in size. A compression force is provided by a second set of elastic properties compressing and receiving at least one of a hot element and a cold element against the user's body part thereby eliminating a second attaching means such as a strap, button, pocket, hook and loop fastener, ridge, or any combination thereof. The wearable device also includes handles configured to help the user pull the wearable device onto the user's body part and bands configured to provide more compression to target areas of the user's body part. The handles provide an easier means of positioning the wearable device on the user's body part. The bands tighten the wearable device proximate to the target areas of the user's body part to prevent at least one of the hot element or the cold element from moving out of position and to stabilize the muscles near the target area. Proximate may be defined as close to or near. As a result, the unrestricted application of a therapeutic modality provides a more effective treatment method because the wearable device conforms to the body and anchors the therapeutic modality precisely over the injury.

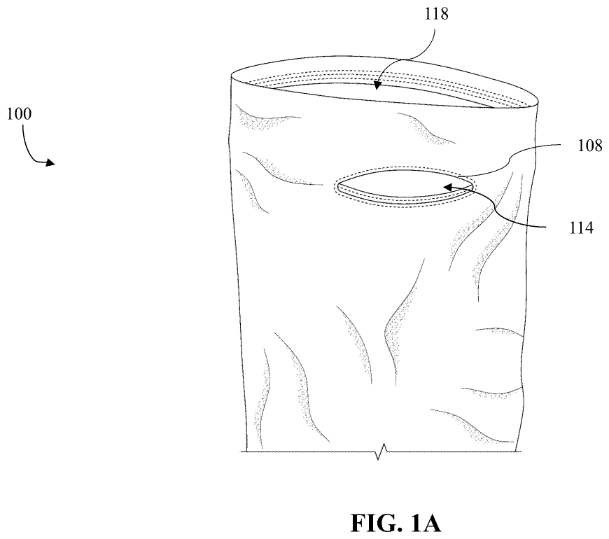

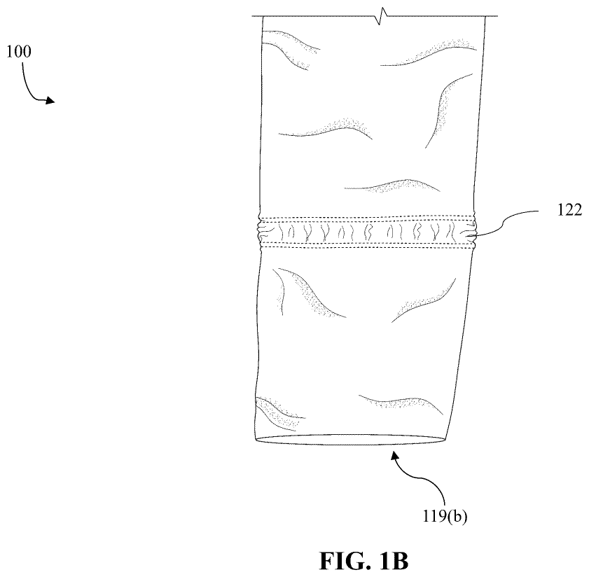

Referring now to the Figures, FIGS. 1A-6 illustrate an apparatus and system for at least one of icing, heating, and compressing a user's body part 101, and will be discussed together for ease of reference. The wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 has a first set of elastic properties and a first tubular shape such that the inner part 105 is configured to fit snugly around a user's body part. The inner part also has an inner part thickness configured to allow the inner part to exchange heat with the user's body part. The purpose of the inner part is to protect the user's skin from at least of the hot element and the cold element when the wearable device is worn. The inner part thickness is configured to allow the hot element or cold element to warm or cool the target area of the user's body part while also preventing the hot element or cold element from directly contacting the user's skin. The outer part 110 includes an outer part upper end 111 and an outer part lower end 112 defining an outer part length. The outer part is comprised of a water-wicking material to draw moisture away from the body and prevent ice from leaking during treatment. The outer part 110 has a second set of elastic properties and a second tubular shape. The first set of elastic properties defines a first resilient force of between 17 mmHg-22 mmHg, and as described more fully below, when the wearable device 100 is in a fully sealed configuration, the second set of elastic properties defines a second resilient force of between 17 mmHg-22 mmHg. Elastic compression is the resilient force exerted by the wearable device on the user's body part. Compression is expressed in millimeters of mercury (mmHg). A higher number is correlated with a higher amount of compression while a lower number correlates with a lower compression.

At least one handle 108 is in attachment with the outer part to allow the user to pull up the wearable device when being worn. The at least one handle is defined by a first handle opening 114 extending through the outer part and the inner part. The handle allows provides a convenient means for the user to pull up the wearable device such that wearable device may be easily positioned on the user's body part. The handle is sized such that the user can grasp the handle with its entire hand. Additionally, because the wearable device may be tight on the user, the handles provide the user with a reliable means of grasping the wearable device to easily pull up the wearable device on the user's body part. In some embodiments. At least one handle may be disposed on opposing sides of the wearable device such that the user may pull up the wearable device evenly using two hands. In other embodiments, the handles may be positioned on any portion of the wearable device allowing the user to pull up the wearable device.

At least one band 122, as shown in FIG. 1B, is disposed on the outer part between the outer part upper end and outer part lower end. The band is looped around the wearable device such that the band is around the body part. The band is in attachment with the outer part but is outside the inner surface of the outer part. For example, the band may be sewn or embedded into the outer part such that the band is not inside the gap between the outer part and the inner part. This prevents the band from obstructing the compartment. The band has a third set of elastic properties that is greater than the first set of elastic properties and the second set of elastic properties. The third set of elastic properties has a third resilient force that is greater than the first resilient force and the second resilient force. The band causes the wearable device to be tighter around the target area of user's body part, where the band is proximate to. The target area may be where therapeutic modalities are positioned. For example, the target area may be where the user requires at least one of the hot element or the cold element. The tightness of the band prevents the hot element or cold element from sliding down and away from the target area. The target area may also be where the user requires more compression. The increased compression helps stabilize the muscles near the target area of the user's body part to help decrease fatigue and/or decrease soreness.

In the present embodiment, the wearable device of the system is a pair of shorts comprising a main body member 116 and a pair of leg sections 117(a), 117(b). The main body member is generally tubular for encircling the hip region of a user. The upper end of the main body member has an opening 118 to accommodate the hip region of a user. The main body member opening is defined by a flap 150 forming a seal at the upper end of the wearable device 100. The flap is defined by the inner part length being greater than the outer part length such that the inner part upper end 106 folds over the outer part upper end 111. When the wearable device 100 is in a fully sealed configuration, the flap 150 extends at least 1.0 inch from a top edge of the upper end of the outer part. The flap prevents water or perspiration from the heat element to escape the upper end of the main body. The lower end of the main body member has a pair of openings 119(a), 119(b) to accommodate the legs of the user and are appropriately sized and positioned so that the legs of the user may comfortably extend through the main body member.

The wearable device may be comprised of different flexible materials such as Lycra, polyester, cotton blend, nylon, similar blends, or any other suitable materials known in the art. More specifically, and without limitation, the material can be moisture absorbent, breathable, stretchable, meshed, or any blend or combination thereof. The material can also be thermoconductive to conduct heat better, or thermoreflective to help keep heat inside the wearable device. Further, the thickness and elasticity of fabric may be varied to accommodate different users and conditions. It should be appreciated that the wearable device can have other shapes and dimensions to accommodate men, women, and children of all sizes, and such variations are within the spirit and scope of the claimed invention.

The wearable device further includes a first attaching means 120 for attaching the inner part lower end 107 to the outer part lower end 112. The first attaching means is configured such that the therapeutic modality does not move below the lower ends. In the present embodiment, the first attaching means 120 comprises stitching the inner part lower end 107 to the outer part lower end 112. A plurality of different stitch patterns can be used including a chain stitch, straight stitch, and zigzag stitch, and such variations are within the spirit and scope of the claimed invention. Additionally, the stitching thread can be comprised of a natural fiber (cotton, wool, silk, linen) or a synthetic fiber (rayon, polyester, nylon). It should also be appreciated that other attachment means can be used to attach the inner part lower end 107 to the outer part lower end 112, including fasteners, snaps, zippers, ties, or any other suitable method known in the art.

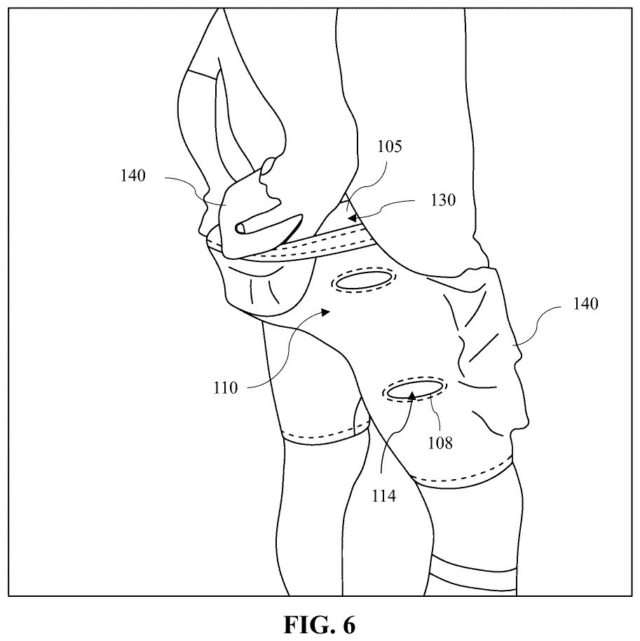

The wearable device further includes a compartment 130 defined by the space between the outer part 110 and the inner part 105, located above the first attaching means 120. It is understood that when the wearable device is worn by the user the inner part and the outer “sandwich” the cold element or hot element that has been inserted in the compartment. The inner layer may also provide an increased amount of friction to further prevent the cold or hot element from moving and facilitates maintaining the cold or hot element in a single position between the inner layer and outer layer unless acted on by a force greater than the inward compression force. The compartment is configured for receiving at least one of a hot element and cold element 140. The present invention improves upon the prior art by allowing the unrestricted application of the therapeutic modality to the user's body. In other words, the therapeutic modality is not limited or restricted to a particular location or region on the user's body. As best shown in FIG. 6, when the wearable device 100 is not in a fully sealed configuration, the therapeutic modality may be positioned and adjusted without removing, changing, or adding layers within the compartment in an unlimited number of positions within the compartment spanning the entire wearable device. As a result, the unrestricted application of the therapeutic modality provides a more effective treatment method because the wearable device conforms to the body and anchors the therapeutic modality precisely over the injury.

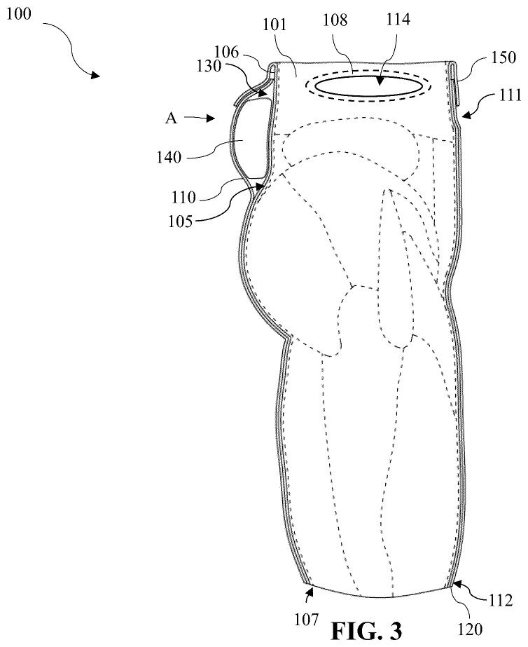

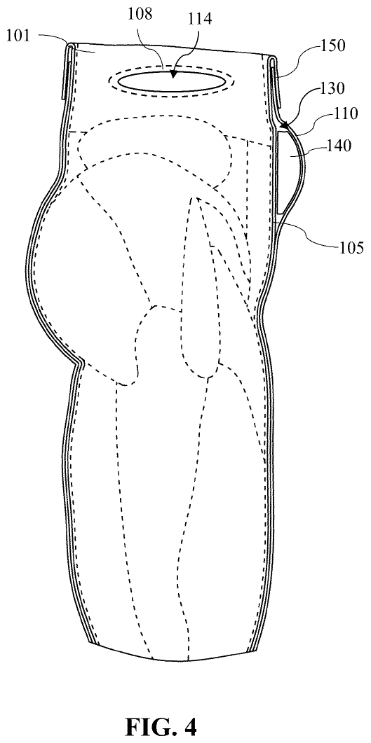

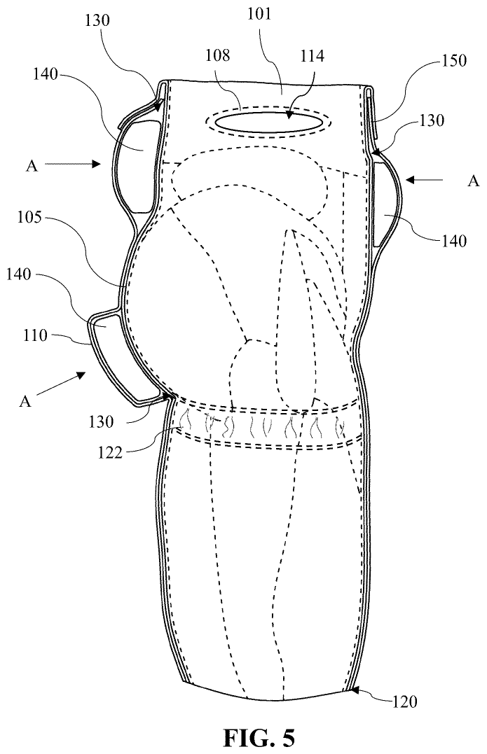

A compression force is provided by the second set of elastic properties compressing and securing at least one of a hot element and cold element against the user's body part 101. A The hot and cold elements may be comprised of crushed ice, commercially available ice and gel packs, hot water bottles, heat pads, and heated stones. However, other devices or apparatus may be used for hot or cold elements that are within the spirit and scope of the present invention. The compression force provides an inward force (in the direction of line A) of at least 17 mmHg against the user's body part, thereby eliminating a second attaching means above the first attaching means 120. The second attaching means being a structure other than the outer part 110 for securing the at least one of the hot element and cold element against the user's body part 101, and may include a strap, button, pocket, hook and look fastener, snap, ridge, or any combination thereof. In other words, the outer part having the second set of elastic properties eliminates the need for unnecessary pockets, straps of fasteners for securing the hot or cold element against the user's body. FIGS. 3-5 illustrates that the wearable device snugly fits the body of the user such that multiple therapeutic modalities located inside the compartment are anchored precisely over the desired area due to the snug fit of the wearable device. For example, in FIG. 3, a cold element 140 is positioned over the lower back (lumbar muscle) of the user's body part 101, and in FIG. 4, a cold element positioned over the anterior wall (abdominal muscle) of the user's body part 101. In FIG. 5, three cold elements are simultaneously positioned over the lower back (lumbar muscle), the anterior wall (abdominal muscle), and posterior pelvic region (gluteus maximus muscle) of the user's body part 101. By eliminating a second attaching means, being structure, above the first attaching means, the wearable device can be worn as any layer of clothing, including as an undergarment or as an over-garment, and permits the user to move, in moderation, without disturbing the precision of treatment. Additionally, by eliminating the second attaching means allows the user to position the hot element or cold element in a much greater amount of locations more easily that the existing prior art.

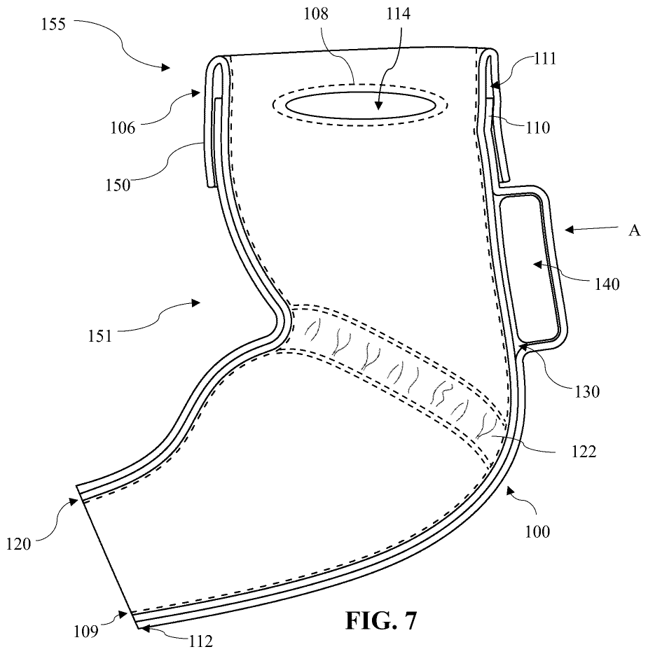

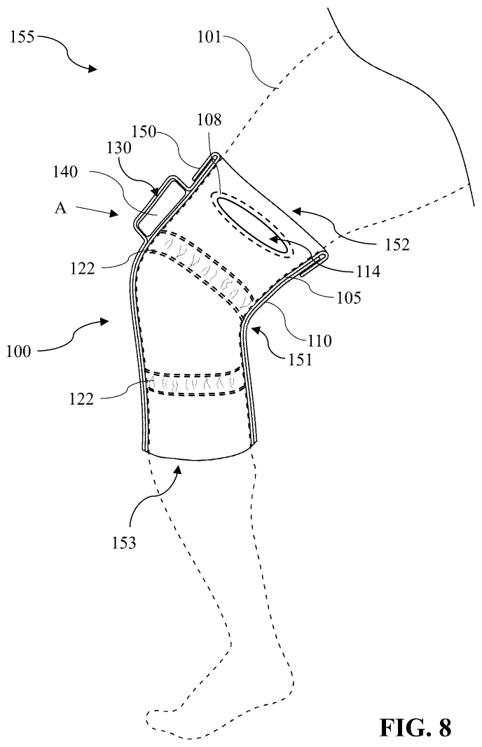

FIGS. 7-8 illustrate a system 155 for at least one of icing, heating, and compressing a user's body part 101 and will be discussed together for ease of reference. The system 155 includes a wearable device 100, and at least one of a hot element and a cold element positioned within a compartment on the wearable device. The wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 is arranged having a first set of elastic properties and a first tubular shape such that the inner part 105 is configured to fit snugly around a user's body part. The outer part 110 includes an outer part upper end 111 and an outer part lower end 112 defining an outer part length. The outer part may be comprised of a water-wicking material to draw moisture away from the body and prevent ice from leaking during treatment. However, it is understood that other types of materials may be used and are within the spirit and scope of the present invention. The outer part 110 has a second set of elastic properties, also configured to fit snugly around a user's body part, and a second tubular shape. The system includes a first set of elastic properties defining a first resilient force of between 17 mmHg-22 mmHg, and as described more fully below, when the wearable device 100 is in a fully sealed configuration, the second set of elastic properties defines a second resilient force of between 17 mmHg-22 mmHg.

In another embodiment, the wearable device is a knee sleeve comprising a main body member 151. The main body member is generally tubular for encircling the knee region of the user's body part 101. The upper end of the main body member has a first opening 152 to accommodate the upper knee region of the user. The first opening is defined by a flap 150 forming a seal at the upper end of the wearable device 100. The flap is defined by the inner part length being greater than the outer part length such that the inner part upper end 106 folds over the outer part upper end 111. When the wearable device 100 is in a fully sealed configuration, the flap 150 extends at least 1.0 inch from a top edge of the upper end of the outer part. The lower end of the main body member has a second opening 153 to accommodate the lower knee region of the user. The first and second openings are appropriately sized and positioned so that the leg of the user may comfortably extend through the first and second opening of the main body member.

The wearable device may be comprised of different flexible materials such as Lycra, polyester, cotton blend, nylon, similar blends, or any other suitable materials known in the art. More specifically, and without limitation, the material can be moisture absorbent, breathable, stretchable, meshed, or any blend or combination thereof. The material can also be thermoconductive to conduct heat better, or thermoreflective to help keep heat inside the wearable device. Further, the thickness and elasticity of fabric may be varied to accommodate different users and conditions. It should be appreciated that the wearable device can have other shapes and dimensions to accommodate men, women, and children of all sizes, and such variations are within the spirit and scope of the claimed invention.

The wearable device further includes a first attaching means 120 for attaching the inner part lower end 107 to the outer part lower end 112. The first attaching means is configured such that the therapeutic modality does not move below the lower ends. The first attaching means may include a strap, button, pocket, hook and loop fastener, ridge, etc. In the present embodiment, the first attaching means 120 comprises stitching the inner part lower end 107 to the outer part lower end 112 such that the outer part surrounds the inner part. A plurality of different stitch patterns can be used including a chain stitch, straight stitch, and zigzag stitch, and such variations are within the spirit and scope of the claimed invention. Additionally, the stitching thread can be comprised of a natural fiber (cotton, wool, silk, linen) or a synthetic fiber (rayon, polyester, nylon). It should also be appreciated that other attachment means can be used to attach the inner part lower end 107 to the outer part lower end 112, including fasteners, snaps, zippers, ties, or any other suitable method known in the art.

The wearable device further includes a compartment 130 defined by the space between the outer part 110 and the inner part 105, located above the first attaching means 120 and positioned outward from the user's body. The space may vary in size across other embodiments. The compartment is configured for receiving at least one of a hot element and cold element 140. As best shown in FIG. 6, when the wearable device 100 is not in a fully sealed configuration, the therapeutic modality may be easily positioned directly over any injured area and adjusted without removing, changing, or adding layers within the compartment in an unlimited number of positions within the compartment spanning the entire wearable device.

A compression force is provided by the second set of elastic properties compressing at least one of a hot element and cold element against the user's body part 101. The hot and cold elements may be comprised of crushed ice, commercially available ice and gel packs, hot water bottles, heat pads, and heated stones. The compression force provides an inward force (in the direction of line A) of at least 17 mmHg against the user's body part, thereby eliminating a second attaching means above the first attaching means 120. As mentioned, above, the second attaching means being a structure other than the outer part 110 for securing the at least one of the hot element and cold element against the user's body part 101, and may include a strap, button, pocket, hook and look fastener, snap, ridge or any combination thereof. FIG. 8 illustrates that the wearable device snugly fits the body of the user's body part 101 such that a therapeutic modality located inside the compartment is anchored precisely over the knee area due to the snug fit of the wearable device. By eliminating a second attaching means above the first attaching means, the wearable device can be worn as any layer of clothing, including as an undergarment or as an over-garment, and permits the user to move, in moderation, without disturbing the precision of treatment. Additionally, the compression force provided by the second set of properties is configured such that the second attaching means is eliminated thereby allowing the user to position the hot element or cold element in a much greater amount of locations more easily that the existing prior art. Additionally as mentioned above the inner part may also provide an amount of friction thereby facilitating prevention of movement of the hot element or cold element when the element is positioned within the compartment and the wearable device is worn by the user.

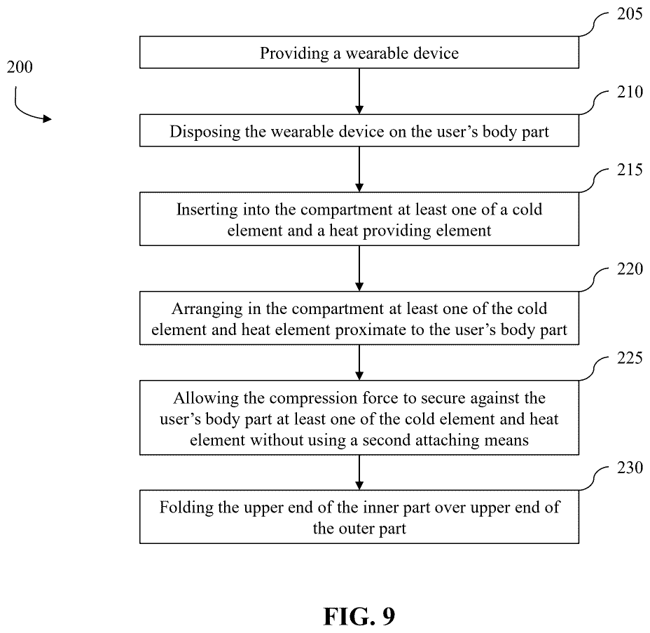

FIG. 9 is a flowchart describing the steps of the method for at least one of icing, heating, and compressing a user's body part, according to an example embodiment of the present invention. The sequence of steps depicted is for illustrative purposes only and is not meant to limit the method in any way as it is understood that the steps may proceed in a different logical order, additional or intervening steps may be included, or described steps may be divided into multiple steps, without detracting from the invention.

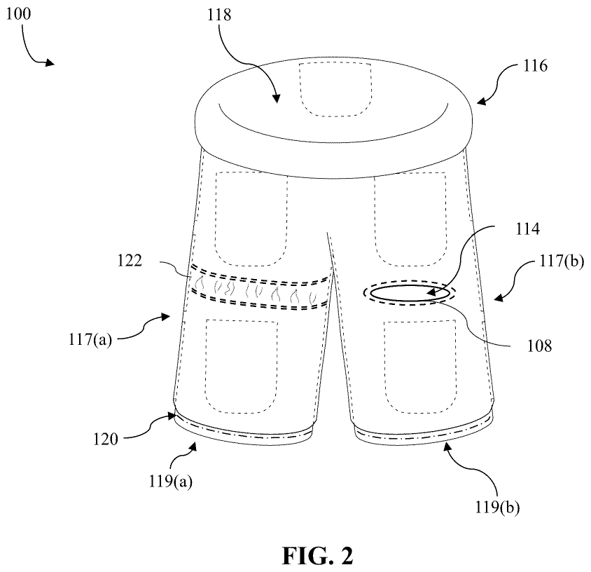

The method 200 begins with step 205, providing a wearable device. As described above, with respect to FIGS. 2-8, the wearable device 100 comprises an inner part 105 and outer part 110. The outer part 110 is arranged with the inner part 105 such that the outer part surrounds the inner part. The inner part 105 includes an inner part upper end 106 and an inner part lower end 107, defining an inner part length. The inner part provides a protective insulation layer between a cold or heat modality and a user's skin. The inner part 105 has a first set of elastic properties and a first tubular shape such that the inner part 105 is configured to fit snugly around a user's body part. The outer part 110 includes an outer part upper end 111 and an outer part lower end 112 defining an outer part length. The outer part may be comprised of a water-wicking material to draw moisture away from the body and prevent ice from leaking during treatment. The outer part 110 has a second set of elastic properties and a second tubular shape. The first set of elastic properties defines a first resilient force of between 17 mmHg 22 mmHg, and as described more fully below, when the wearable device 100 is in a fully sealed configuration, the second set of elastic properties defines a second resilient force of between 17 mmHg-22 mmHg. It is important that the second resilient force be sufficient such that the compression force or inward force provided by the second set of elastic properties is sufficient such that the hot element and cold element is maintained where it is positioned and such that the need for the second attaching means, being structure, is eliminated. A flap is defined by the inner part length being greater than the outer part length such that the inner part upper end 106 folds over the outer part upper end 111. When the wearable device 100 is in a fully sealed configuration, the flap 150 extends at least 1.0 inch from a top edge of the upper end of the outer part. The wearable device further includes a first attaching means 120 arranged for attaching the inner part lower end 107 to the outer part lower end 112 such that the outer part surrounds the inner part. The first attaching means is configured such that the therapeutic modality does not move below the lower ends. A compartment 130 is defined by the space between the outer part 110 and the inner part 105, located above the first attaching means 120. The compartment is configured for receiving at least one of a hot element and cold element 140. A compression force is provided by the second set of elastic properties compressing at least one of a hot element and cold element against the user's body part 101. As shown in FIGS. 3-5, the compression force provides an inward force (in the direction of line A) of at least 17 mmHg against the user's body part, thereby eliminating a second attaching means above the first attaching means 120. A first handle opening and a second handle opening extends through the outer part and the inner part located opposing sides. The first handle opening and the second handle opening are disposed on opposing sides of the wearable device. For example, when the wearable device is worn, the first handle opening may be on the right side of the user's body part, and the second handle opening may be on the left side of the ser's body part. The wearable device may include additional handle openings, not located on opposing sides, to pull up different parts of the wearable device. The wearable device also includes at least one band 122 having a third set of elastic properties that is greater than the first set of elastic properties of the inner part and the second set of elastic properties of the outer part. The band may be positioned so that it is outside the inner surface of the outer part so that the band does not unnecessarily obstruct the compartment. In other words, the band is such that it provides a compressive force without adding elements within the compartment (which is between the inner surface of the outer layer and the outer surface of the inner layer). The band may be of an elastic material and other types of materials may be used and are within the spirit and scope of the present invention.

In step 210, the method includes disposing the wearable device on the user's body part. Disposing the wearable device includes pulling the wearable device onto the user's body part, ideally using the first handle opening and the second handle opening disposed on opposing sides. The user's body is positioned inward from a channel from the inner part, and the compartment is positioned outward from the user's body part. The at least one band 122 is disposed proximate to a target area of the user. The target area of the user may be the portion of the user's body part that may need more compression. The target area of the user may also be the portion where the user requires the hot element or the cold element such that the band is disposed directly below the compartment containing the hot element or the cold element. The band provides more compression to prevent the hot element or cold element, one inserted into the compartment, from sliding down between the inner part and outer part where the band is disposed. The band may be stitched to the inner side of the outer part. In one embodiment may be a looped element or continuous looped shaped elastic band that is sticked to the inward facing surface of the outer part so that the band provides a compressive force on a user's body.

As shown in FIGS. 2-6, in one embodiment, the wearable device is a pair of shorts comprising a main body member 116 and a pair of leg sections 117(a), 117(b). The main body member is generally tubular for encircling the hip region of a user. The upper end of the main body member has an opening 118 to accommodate the hip region of a user. The lower end of the main body member has a pair of openings 119(a), 119(b) to accommodate the legs of the user and are appropriately sized and positioned so that the legs of the user may comfortably extend through the main body member.

As shown in FIGS. 7-8, in another embodiment, the system includes a wearable device being a knee sleeve comprising a main body member 151. The main body member is generally tubular for encircling the knee region of the user's body part 101. The upper end of the main body member has a first opening 152 to accommodate the upper knee region of the user. The lower end of the main body member has a second opening 153 to accommodate the lower knee region of the user. The first and second openings are appropriately sized and positioned so that the leg of the user may comfortably extend through the first and second opening of the main body member.

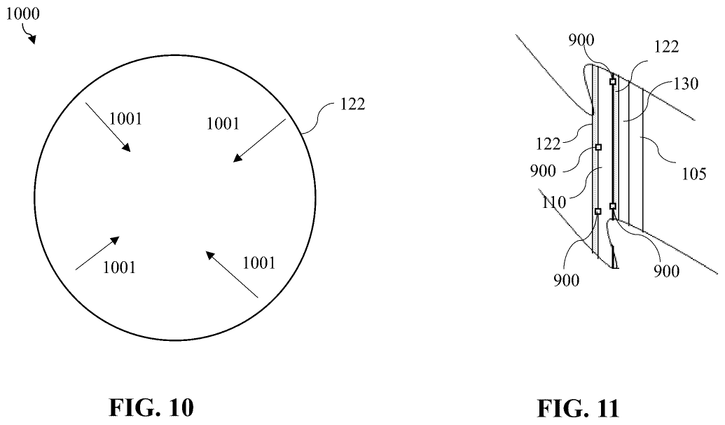

Referring to FIGS. 10 and 11, FIG. 10 is a top view of a diagram of a band, according to an example embodiment and FIG. 11 is a partial cross-sectional view of a pair of bands (122) attached to the outer part 110. The band is looped to wrap around the body part when the wearable device is worn. In one embodiment, the band may be attached by stitching 900 to the inward facing side of the outer part so that hot and cold elements can easily be positioned inward from the band inside the compartment 130. The thickness of the band is configured to be thin enough to not obstruct the compartments. The band should not obstruct the compartment because the user may decide to push the hot element or cold element further below the band. Additionally, a second band (122) may be attached to the wearable device by stitching 900 to the surrounding outward facing part of the outer part so that hot and cold elements inserted can easily be positioned inward from the band inside the compartment 130. In other embodiments, the bands may be attached to the outer part via other processes. The thickness of the second band is also configured to be thin enough such the second band may be seamlessly disposed on the wearable device. As mentioned above and shown in FIG. 10, the bands 1000 apply a compressive force in the direction of arrowed lines 1001 such that when the bands are on a user's body the compressive force prevents cold and hot elements from moving downward and falling out of position. Because the bands have the third set of elastic properties that are tighter than the elastic properties of the outer part and inner part, the compressive force of the bands tightens the area of the wearable device more where the bands are positioned. Therefore, the bands push the outer part against the inner part to tighten the gap between the inner part and the outer part. While the wearable device is worn, the tightened gap prevents the inserted hot element or cold element from moving below the area of the wearable device where the bands are positioned. The increased compression may also provide more stabilization to the user's muscles where the bands are positioned. In other embodiments, the band may be a looped drawstring disposed on the outward facing part of the outer part. The drawstring would provide the same compressive force in the direction of the arrowed lines 1001 configured to tighten the gap between the outer part and the inner part. Additionally, the band may have a height (top of band to bottom of ban) substantially less than the length (from the upper end to the lower end) of the outer part and inner part as illustrated in the figures.

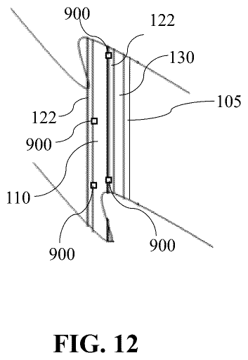

FIG. 12 further illustrates that the thickness of the outer part 110 has an outer part thickness that is greater than the thickness of the inner part. Inner part may be a thickness of 200 grams per square meter (GSM), which is lighter and thinner weight than the outer part, which may allow for easy on application, allow for smooth sliding surface to receive ice, and frostbite insulation protection when the device is worn by the user. The outer part 110 may comprise 250 grams per square meter (GSM), which is heavier or thicker material than the inner part 105 for clamping capability to hold ice in place against a user in unlimited positions throughout the sleeve (capacity, region, etc.). However, other ranges may be used and are within the spirit and scope of the present invention as long as the outer part 110 has a greater weight or thickness and provide a greater compression force when the device is worn by the user than the inner part 105. Each sleeve may comprise of at least Nylon which produces a stronger, less stretchy material. It is understood that thickness may also be used to refer to the weight of fabric. In short, the weight of the fabric on the outer part may be greater than the weight of the fabric of the inner part.

In step 215, the method includes inserting into the compartment at least one of a cold element and a heat providing element. The hot and cold elements may be comprised of crushed ice, commercially available ice and gel packs, hot water bottles, heat pads, and heated stones. As best shown in FIG. 6, when the wearable device 100 is not in a fully sealed configuration, the at least one of the cold element and heat element may be positioned and adjusted without removing, changing or adding layers within the compartment in an unlimited number of positions within the compartment spanning the entire wearable device. Additionally, the present embodiment allows the user to insert and position a hot element or cold element proximate to a user's body and within the wearable device in numerous positions without being limited by use of a fastener such as a hook, button, flap, snap, ridge, etc.

In step 220, the method includes arranging in the compartment at least one of the cold element and heat element proximate to the user's body part. For example, in FIG. 3, a cold element 140 is positioned over the lower back (lumbar muscle) of the user's body part 101, and in FIG. 4, one cold element positioned over the anterior wall (abdominal muscle) of the user's body part 101. In FIG. 5, three cold elements are simultaneously positioned over the lower back (lumbar muscle), the anterior wall (abdominal muscle), and posterior pelvic region (gluteus maximus muscle) of the user's body part 101. Force (opposing the inward force in the direction of line A) may be applied to the outer part to stretch the outer part and enlarge the compartment so that the hot element or cold element may be positioned into the compartment. After positioning the hot element or cold element into the compartment, then the force stretching the outer part to enlarge the compartment may be removed so that the compression force provided by the outer layer compresses the hot element or cold element against the user's body.

In step 225, the method includes allowing the compression force to secure against the user's body part at least one of the cold element and heat element without using a second attaching means. The heat element and/or cold element are unrestrictedly positioned above the first attaching means such that the heat element and cold element are removably positioned within the compartment. The heat element and/or cold element may be removed from the compartment by the user. This allows the user to replace or flip the heat element and/or cold element. The second attaching means is a structure other than the outer part 110 for securing the at least one of the hot element and cold element against the user's body part 101, and may include a strap, button, pocket, hook and look fastener, snap, ridge, or any combination thereof. As shown in FIGS. 3-5, the compression force provides an inward force (in the direction of line A) of at least 17 mmHg against the user's body part, thereby eliminating a second attaching means above the first attaching means 120. By eliminating a second attaching means above the first attaching means, the wearable device can be worn as any layer of clothing, including as an undergarment or as an over-garment, and permits the user to move, in moderation, without disturbing the precision of treatment. Additionally, by eliminating the second attaching means allows the user to position the hot element or cold element in a much greater amount of locations more easily than the existing prior art.

In step 230, the method includes folding the upper end of the inner part over the upper end of the outer part so that the flap is positioned outward from the outer part creating the seal. As discussed above, the flap is defined by the inner part length being greater than the outer part length such that the inner part upper end 106 folds over the outer part upper end 111. Thus, when the wearable device 100 is in a fully sealed configuration, the flap 150 extends at least 1.0 inch from a top edge of the upper end of the outer part.

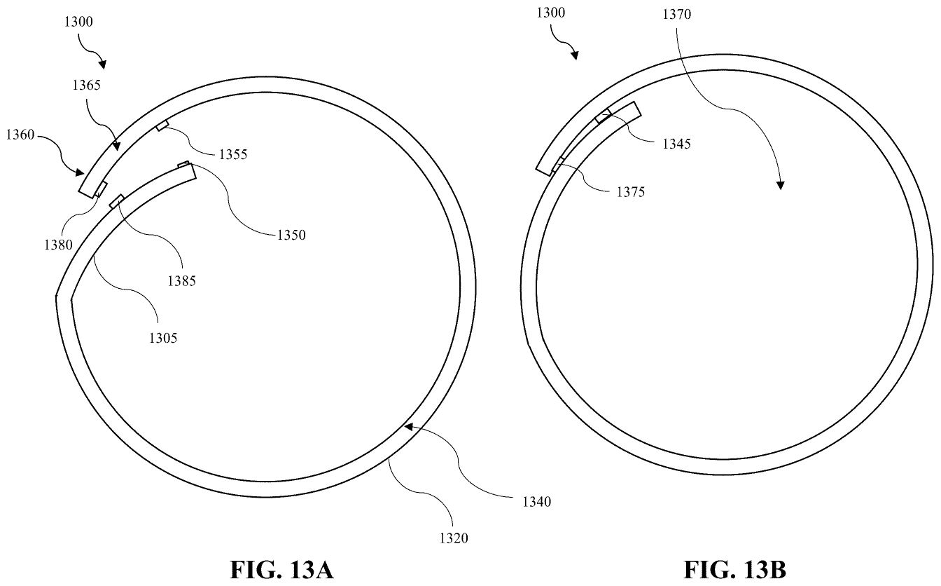

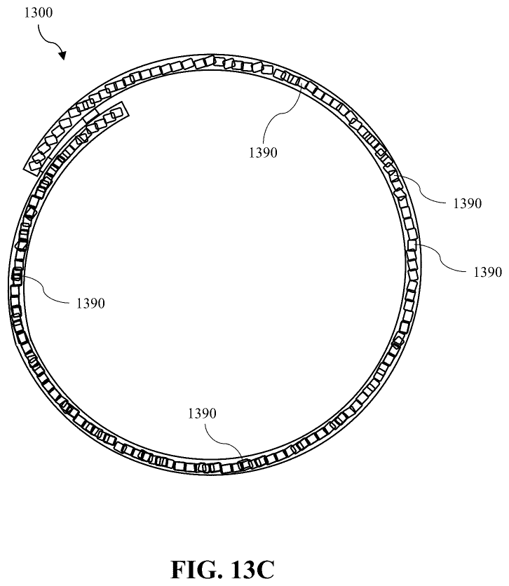

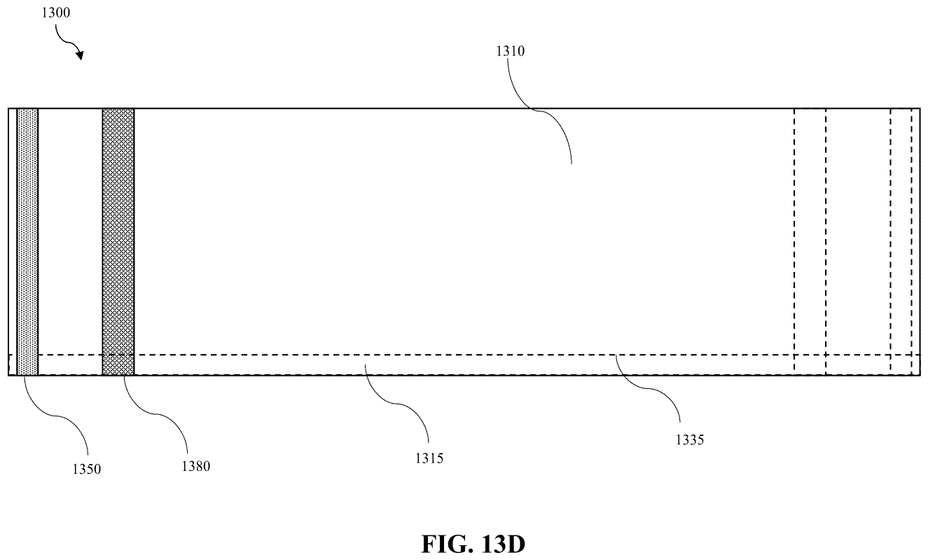

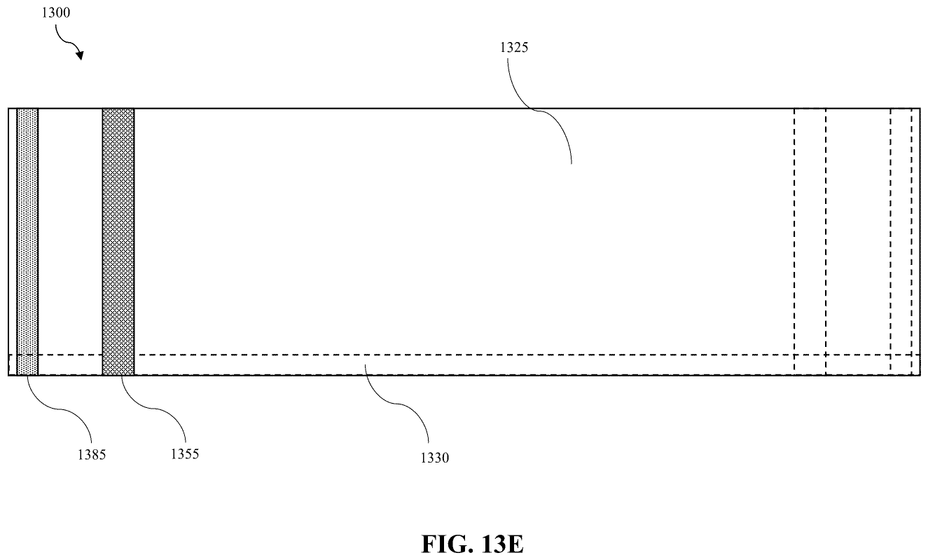

Referring now to the Figures, FIGS. 13A-13E illustrate a wearable device 1300 for at least one of icing, heating, and compressing a user's body part, and will be discussed together for ease of reference.

The wearable device 1300 comprises an inner part 1305 and an outer part 1320 is a flexible, elastic layer that forms the interior surface of the wearable device. It is designed to be in direct contact with the user's body, providing comfort and protection. The inner part acts as a barrier between the user's skin and the ice or heat element inserted into the compartment. Its primary function is to prevent direct contact with the cold or hot element, thereby avoiding potential skin damage such as frostbite or burns. At the same time, the inner part facilitates efficient thermal transfer, allowing the therapeutic benefits of cold or heat therapy to reach the affected area. The material of the inner part is typically moisture-wicking, breathable, and elastic to ensure a snug fit. Suitable materials include Lycra, spandex, polyester blends, or other synthetic fabrics that provide stretchability and comfort. The inner part may also have a smooth, low-friction surface to facilitate easy insertion and adjustment of ice or heat packs within the compartment. The inner part 1305 further comprises an inner part upper end 1310 and an inner part lower end 1315, defined by a first attaching means 1335. The inner part upper end 1310 forms the upper boundary of the inner part. This upper end extends to the highest edge of the wearable device and plays a critical role in forming a secure seal at the top of the wrap. When the extension flap folds over the upper end of the outer part, the inner part upper end contributes to sealing the compartment, preventing ice or heat elements from slipping out of the top. This feature helps contain moisture and maintains the integrity of the compartment. The upper end is often reinforced to provide additional durability, especially at the points where it interfaces with the outer part and the second attaching means. The inner part upper end ensures that the therapeutic element remains securely positioned, even during movement or physical activity. The inner part lower end 1315 defines the lower boundary of the inner part. This lower end is securely attached to the outer part lower end using the first attaching means, such as stitching, bonding, or other fastening techniques. The attachment of the lower end creates a sealed compartment, ensuring that ice or heat elements do not leak or slip out through the bottom. The inner part lower end plays a crucial role in maintaining the positioning of the therapeutic elements, providing stability, and preventing displacement. This feature ensures consistent compression and temperature application during use. The lower end may be reinforced with additional stitching or fabric layers to enhance durability and withstand repeated use or stretching.

The outer part 1320 is the exterior layer of the wearable device that surrounds the inner part. It is designed to provide structural integrity, compression, and containment for the therapeutic elements inserted into the compartment. The outer part holds the ice or heat element firmly against the inner part, ensuring effective compression against the user's body. This layer is typically made from durable, elastic, and water-resistant materials such as neoprene, nylon, or polyester blends. The outer part's elasticity ensures a snug fit, maintaining consistent pressure and preventing the therapeutic elements from shifting during use. Additionally, the outer part helps wick away moisture, preventing leaks and keeping the device dry and comfortable. The outer part is designed to withstand the rigors of physical activity while maintaining its compressive and structural properties. The outer part 1320 further comprises an outer part upper end 1325 and an outer part lower end 1330, defined by a first attaching means 1335. The outer part upper end 1325 forms the upper boundary of the outer part and aligns with the inner part upper end. This upper end provides structural support for the extension flap, helping to create a secure seal at the top of the compartment. The outer part upper end works with the second attaching means to ensure that the therapeutic element remains securely positioned within the compartment. It also helps maintain the device's overall structure and compression, preventing the therapeutic elements from slipping out through the top. Reinforcements or additional stitching at the upper end can enhance durability and ensure that the device can withstand repeated use. The outer part lower end 1330 defines the lower boundary of the outer part. It is securely attached to the inner part lower end using the first attaching means. This attachment seals the bottom of the compartment, preventing the therapeutic element from escaping or leaking out. The outer part lower end provides structural integrity and ensures that the compression force remains consistent throughout the device. By sealing the lower portion, this feature ensures that the therapeutic element remains in place, delivering uniform compression and temperature application. The lower end may be reinforced to enhance durability and withstand the stretching forces applied during use.

The first attaching means 1335 is a mechanism for securely connecting the inner part lower end 1315 to the outer part lower end 1330. This attachment can be achieved through various methods such as stitching, bonding, or other fastening techniques. Common examples include chain stitching, straight stitching, zigzag stitching, or adhesive bonding. The first attaching means 1335 creates a defined lower boundary for the compartment, ensuring that the therapeutic element cannot slip out from the bottom. This attachment maintains the structural integrity of the device and helps distribute compression evenly. Reinforced stitching or additional fabric layers can be used to enhance durability and ensure that the attachment withstands repeated use and stretching.

The wearable device 1300 further comprises a compartment 1340 defined by a space between the inner part 1305 and the outer part 1320 and above the first attaching means 1335. The compartment 1340 is designed to hold ice 1390 or heat elements, allowing the user to insert therapeutic elements for targeted treatment. The compartment provides 360-degree coverage, ensuring uniform compression and temperature application around the body part. The inner part 1305 protects the skin from direct contact with the therapeutic element, while the outer part 1320 applies compressive force to hold the element in place. The compartment's size and shape can accommodate various therapeutic elements, such as ice 1390, ice packs, gel packs, or heat packs. The compartment design ensures that the therapeutic element remains securely positioned during movement or physical activity.

The wearable device 1300 even further comprises a second attaching means 1345, wherein a first part 1350, of the second attaching means 1345 is in attachment with an end portion 1360 of at least the inner part 1305 and the outer part 1320, wherein a second part 1355 of the second attaching means 1345 is in attachment with at least one of the inner part 1305 and outer part 1320 away from the end portion 1360. The second attaching means 1345 is a closure mechanism that secures the wearable device around a user's body part. This attaching means typically consists of hook-and-loop fasteners (Velcro), but may also include straps, buckles, adhesives, clips, or other fastening methods. The second attaching means 1345 comprises a first part 1350, which is attached to an end portion of either of the inner and outer parts. The end portion 1360 provides structural support for the closure mechanism, ensuring that the wrap remains securely fastened during use. The end portion may be reinforced to enhance durability and withstand repeated use. It plays a critical role in maintaining the integrity of the second attaching means, ensuring that the wrap stays in place and provides consistent compression and temperature application. The end portion 1360 is located at an edge or terminal section of the wearable device. The second attaching means 1345 further comprises a second part 1355, which is attached to another section of the inner or outer part away from the end portion. This design allows the wrap to be securely fastened and adjusted for a snug fit, ensuring consistent compression and positioning of the therapeutic element. The second attaching means enables easy application and removal of the wrap while providing a reliable and customizable fit.

When the second attaching means 1345 is secured, an extension flap 1365 extends beyond the second attaching means 1345 on at least one of the inner part 1305 and outer part 1320. The extension flap 1365 is a fabric flap that extends beyond the second attaching means on at least one of the inner or outer parts. The extension flap 1365 is positioned at the end of the wearable device opposite the end portion 1360. This flap extends beyond the second attaching means 1345, ensuring that the compartment within the wearable device is completely enclosed when the device is wrapped around the user's body part. The extension flap provides additional material that folds over the upper edge of the device, contributing to a secure seal and preventing the ice 1390 or heat element from escaping through the top. When the wearable device is secured around a body part, the extension flap effectively overlaps with the area covered by the second attaching means, ensuring that the compartment is enclosed from all sides. This design feature guarantees 360-degree coverage of the compartment, meaning that the therapeutic element (e.g., ice, gel pack, or heat element) is uniformly positioned around the entire circumference of the body part. This complete coverage allows for consistent compression and temperature application across the entire treated area. The extension flap also enhances the structural integrity of the wearable device, helping to maintain the position of the therapeutic element within the compartment during movement or physical activity. By extending beyond the second attaching means, the flap ensures that the closure mechanism remains secure, and the therapeutic element remains properly enclosed, providing effective and uninterrupted treatment.

The wearable device 1300 further comprises a tubular body having a central opening 1370 defined by the first part 1350 removably attached to the second part 1355 of the second attaching means 1345. This tubular structure allows the wrap to encircle the user's body part completely, such as the knee, elbow, or wrist. The central opening 1370 is surrounded by the compartment, allowing for 360-degree compression and temperature application. This design ensures that the therapeutic element is uniformly applied to the entire area covered by the wrap, enhancing the effectiveness of the treatment. The tubular body structure provides stability and consistent pressure, making the wrap suitable for various applications. The compartment 1340 surrounds at least a portion of the central opening 1370, allowing the therapeutic element to be applied uniformly around the entire area. This design ensures that compression and temperature application are consistent throughout the wrap. The compartment's placement around the central opening allows for targeted treatment of the affected area, providing relief and promoting healing.

The wearable device 1300 may further comprise a third attaching means 1375. The third attaching means 1375 is an additional fastening mechanism designed to secure the extension flap 1365 to the wearable device 1300. This third attaching means operates similarly to the second attaching means 1345 but serves the specific purpose of maintaining the extension flap in a fixed position after the wrap has been applied. It ensures the compartment remains fully enclosed and the therapeutic element is held securely in place during use. The third attaching means 1375 comprises a first part 1380 and a second part 1385. The first part 1380 of the third attaching means is attached to the extension flap 1365, typically at the end of the flap that extends beyond the second attaching means 1345. This part can be a hook, Velcro loop, snap, button, adhesive, or another fastening element. The second part 1385 of the third attaching means is affixed to either the inner part 1305 or the outer part 1320 of the wearable device 1300. This placement allows the extension flap to be folded over and secured tightly against the device, ensuring a snug and sealed fit. The third attaching means 1375 comprises two parts: a first part 1380 and a second part 1385. The first part 1380 of the third attaching means is attached to the extension flap 1365, typically at the end of the flap that extends beyond the second attaching means 1345. This part can be a hook, Velcro loop, snap, button, or another fastening element. The second part 1385 of the third attaching means is affixed to either the inner part 1305 or the outer part 1320 of the wearable device 1300. This placement allows the extension flap to be folded over and secured tightly against the device, ensuring a snug and sealed fit.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A wearable device for at least one of icing, heating, and compressing a user's body part, the wearable device comprising:
an inner part having an inner part upper end and an inner part lower end;
an outer part having an outer part upper end and an outer part lower end;
a first attaching means for attaching the inner part lower end to the outer part lower end;
a compartment defined by a space between the outer part and the inner part and above the first attaching means;
a second attaching means, wherein a first part of the second attaching means is in attachment with an end portion of at least the inner part and the outer part, wherein a second part of the second attaching means is in attachment with at least one of the inner part and the outer part away from the end portion;
an extension flap that extends beyond the second attaching means on at least one of the inner part and the outer part; and
a tubular body having a central opening defined by the first part removably attached to the second part of the second attaching means;
wherein at least a portion of the compartment surrounds all of the central opening as the extension flap extends beyond the second attaching means.

2. The wearable device of claim 1, wherein the inner part comprises a moisture-wicking, breathable, and elastic material selected from a group consisting of Lycra, spandex, and polyester blends.

3. The wearable device of claim 1, wherein the first attaching means comprises stitching selected from a group consisting of chain stitching, straight stitching, and zigzag stitching.

4. The wearable device of claim 1, wherein the second attaching means comprises a hook-and-loop fastener.

5. The wearable device of claim 1, wherein a third attaching means comprises a fastening mechanism selected from a group consisting of hook-and-loop fastener, snap, button, and adhesive.

6. The wearable device of claim 1, wherein the outer part comprises a water-resistant elastic material selected from a group consisting of neoprene, nylon, and polyester blends.

7. The wearable device of claim 1, wherein the compartment comprises a shape and dimensions configured to retain a therapeutic element selected from a group consisting of an ice pack, a gel pack, and a heat pack.

8. The wearable device of claim 1, wherein the compartment is annular and circumferentially disposed around the central opening.

9. The wearable device of claim 1, wherein the extension flap comprises a terminal portion extending past the second attaching means and overlapping a portion of the inner part.

10. The wearable device of claim 1, wherein the extension flap is secured to the wearable device using a third attaching means comprising a first part disposed on the extension flap and a second part disposed on the portion of the inner part.
